(19) United States
(12) Patent Application Publication
(10) Pub. No.: US 2011/0257385 A1
(43) Pub. Date: Oct. 20, 2011

(54) METHODS FOR FLIP-STRAND IMMOBILIZING AND SEQUENCING NUCLEIC ACIDS

(75) Inventors: KEVIN MCKERNAN, MARBLEHEAD, MA (US); ALAN BLANCHARD, MIDDLETON, MA (US); GERALD ZON, SAN CARLOS, CA (US); KAI LAO, PLEASANTON, CA (US); NEIL STRAUS, EMERYVILLE, CA (US); EUGENE SPIER, LOS ALTOS, CA (US); CAIFU CHEN, PALO ALTO, CA (US)

(73) Assignee: LIFE TECHNOLOGIES CORPORATION, CARLSBAD, CA (US)

(21) Appl. No.: 13/033,534

(22) Filed: Feb. 23, 2011

Related U.S. Application Data
(60) Provisional application No. 61/307,156, filed on Feb. 23, 2010.

(51) Int. Cl. C07H 21/00; C12P 19/34

(52) U.S. Cl. 536/24.33; 435/91.5; 435/91.53

(57) ABSTRACT

Provided herein are compositions, materials, methods and kits for immobilizing a template polynucleotide in a first orientation, and immobilizing a complementary sequence of the template polynucleotide in an orientation that is flipped compared to the orientation of the template polynucleotide. Provided herein are adaptive oligonucleotides that can be used in various nucleic acid manipulations to generate immobilized complement polynucleotides that are flipped in orientation compared to the orientation of the immobilized template polynucleotides.

This application claims the filing date benefit of U.S. Provisional Application No. 61/307,156, filed on Feb. 23, 2010. The contents of each foregoing patent applications are incorporated by reference in their entirety.

Throughout this application various publications are referenced. The disclosures of these publications in their entireties are hereby incorporated by reference into this application in order to more fully describe the state of the art to which this invention pertains.

### FIELD

This disclosure relates generally to immobilization of polynucleotides in various orientations, including orientations that are flipped or reversed relative to each other. The disclosure also relates to sequencing polynucleotides in various orientations, including orientations that are flipped or reversed relative to each other

### BACKGROUND

Upon completion of the Human Genome Project, the focus of the sequencing industry has shifted to finding higher throughput and/or lower cost sequencing technologies with increased accuracy, sometimes referred to as next generation sequencing technologies. In making sequencing higher throughput and/or less expensive, the technology can be made more accessible for sequencing. These goals can be reached through the use of sequencing platforms and methods that provide sample preparation for larger quantities of samples, sequencing larger numbers of samples, sequencing and/or preparation of samples of increased complexity, and/or a high volume of information generation and analysis in a short period of time. Various methods, such as, for example, sequencing by synthesis, sequencing by hybridization, and sequencing by ligation are evolving to meet these challenges.

Sequencing in commercially available systems such as Sanger CE-sequencing, Roche 454 Pyrosequencing, and Illumina Sequencing-by-Synthesis, that extend the 3′ end of a primer generally proceeds from the 3′ end to the 5′ end of a template nucleic acid. Sometimes it is desirable to obtain sequence information in the reverse direction, i.e., from the 5′ end to the 3′ end of the template nucleic acid. Sequencing templates in either or both the forward and reverse direction using the aforementioned commercially available methods requires multiple different sets of reagents and other materials, including, for example, different enzymes and buffers. Performing either or both forward sequencing and reverse sequencing of a given template therefore can double the number and type of reagents and other materials used during sequencing, making the sequencing process more complex.

### SUMMARY

In some aspects, this disclosure provides compositions, materials, methods and kits useful for immobilizing and/or obtaining sequence data for nucleic acids in various orientations. In some embodiments, a polynucleotide can be immobilized in either 3′-to-5′ and 5′-to'3′ directions and sequence information can be obtained in either 3′-to-5′ and 5′-to'3′ directions, or both. An immobilized polynucleotide can be a template polynucleotide or a polynucleotide having a sequence complementary to a template polynucleotide (i.e., complement polynucleotide).

Methods are provided for immobilizing a template polynucleotide in an orientation that is flipped or reversed compared to the orientation of a complement polynucleotide. The flipped orientation of a complement polynucleotide can permit the use of the same reagents and other materials for performing sequencing processes or reactions on both strands, and can permit the same type of primer extension or sequencing reactions on the template polynucleotide and the complement polynucleotide.

Also provided herein are adaptive oligonucleotides that can be used to generate immobilized complement polynucleotides that are flipped or reversed in orientation compared to the orientation of immobilized template polynucleotides. Adaptive oligonucleotides can include at least one functional sequence or site in any combination and in any order. Functional sequences and sites can include a variety of sequences and sites such as cleavage susceptible sites, cleavage resistant sites, priming sequences, cross-linking sequences, triple-strand forming sequences, restriction endonuclease recognition sequences, nicking endonuclease recognition sequences and the like. Functional sequences or sites can permit manipulation of adaptive oligonucleotides and polynucleotides joined thereto or thereon, to generate a complementary sequence of the template polynucleotide with an orientation that is flipped or reversed compared to the template oligonucleotide.

In some embodiments, adaptive oligonucleotides can fold into one or more secondary structures, such as a U-shaped or hairpin structures. In some embodiments, the adaptive oligonucleotides can be joined to a template polynucleotide or a complement polynucleotide.

Related compositions, materials, methods, and kits are also provided.

### BRIEF DESCRIPTION OF THE DRAWINGS

FIGS. 1A-1D are schematic depictions of a non-limiting embodiment of a flip strand sequencing method.

FIGS. 2A-2G are schematic depictions of a non-limiting embodiment of a flip strand sequencing method.

FIG. 3 is a schematic depiction of a non-limiting embodiment of a flip strand sequencing method.

FIGS. 4A-4F are schematic depictions of a non-limiting embodiment of a flip strand sequencing method.

FIGS. 5A-5F are schematic depictions of a non-limiting embodiment of a flip strand sequencing method.

It is to be understood that the figures are not drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts.

### DESCRIPTION

The section headings used herein are for organizational purposes only and are not to be construed as limiting the described subject matter in any way. All literature and similar materials cited in this application, including but not limited to, patents, patent applications, articles, books, treatises, and internet web pages are expressly incorporated by reference in their entirety for any purpose. When definitions of terms in incorporated references appear to differ from the definitions provided in the present teachings, the definition provided in the present teachings shall control. It will be appreciated that there is an implied “about” prior to the temperatures, concentrations, times, etc. discussed in the present teachings, such that slight and insubstantial deviations are within the scope of the present teachings. In this application, the use of the singular includes the plural unless specifically stated otherwise. Also, the use of “comprise”, “comprises”, “comprising”, “contain”, “contains”, “containing”, “include”, “includes”, and “including” are not intended to be limiting. It is to be understood that both the foregoing general description and the following detailed description are exemplary and explanatory only and are not restrictive of the present teachings.

Unless otherwise defined, scientific and technical terms used in connection with the present teachings described herein shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well known and commonly used in the art. Standard techniques are used, for example, for nucleic acid purification and preparation, chemical analysis, recombinant nucleic acid, and oligonucleotide synthesis. Enzymatic reactions and purification techniques are performed according to manufacturer's specifications or as commonly accomplished in the art or as described herein. The techniques and procedures described herein are generally performed according to conventional methods well known in the art and as described in various general and more specific references that are cited and discussed throughout the instant specification. See, e.g., Sambrook et al., Molecular Cloning: A Laboratory Manual (Third ed., Cold Spring Harbor Laboratory Press, Cold Spring Harbor, N.Y. 2000). The nomenclatures utilized in connection with, and the laboratory procedures and techniques described herein are those well known and commonly used in the art.

As used herein, the phrase “next generation sequencing” refers to sequencing technologies having increased throughput as compared to traditional Sanger- and capillary electrophoresis-based approaches, for example with the ability to generate hundreds of thousands of relatively small sequence reads at a time. Some examples of next generation sequencing techniques include, but are not limited to, sequencing by synthesis, sequencing by ligation, and sequencing by hybridization. Examples of next generations sequencing methods include pyrosequencing as used by 454 Corporation, Illumina's Solexa system, and the SOLiD™ (Sequencing by Oligonucleotide Ligation and Detection) system developed by Applied Biosystems (now part of Life Technologies, Inc.).

The term “template polynucleotide”, “template nucleic acid”, “target polynucleotide”, and variations refer to a nucleic acid strand that serves as the basis nucleic acid for generating a complementary nucleic acid strand. The sequence of the template polynucleotide can be complementary to the sequence of the complementary strand. The template polynucleotide can be subjected to nucleic acid analysis, including sequencing and composition analysis. The template polynucleotides can be isolated in any form including chromosomal, genomic, organellar (e.g., mitochondrial, chloroplast or ribosomal), recombinant molecules, cloned, amplified, cDNA, RNA such as precursor mRNA or mRNA, oligonucleotide, or any type of nucleic acid library. The target nucleic acid molecules may be isolated from any source including from organisms such as prokaryotes, eukaryotes (e.g., humans, plants and animals), fungus, and viruses; cells; tissues; normal or diseased cells or tissues, body fluids including blood, urine, serum, lymph, tumor, saliva, anal and vaginal secretions, amniotic samples, perspiration, and semen; environmental samples; culture samples; or synthesized nucleic acid molecules prepared using recombinant molecular biology or chemical synthesis methods. The template polynucleotide can be chemically synthesized to include any type of nucleic acid analog.

The term “complement polynucleotide”, “polynucleotide having a sequence complementary to a template polynucleotide”, and variations refers to a nucleic acid strand that can be generated using a template polynucleotide as a basis nucleic acid. The complement polynucleotide can have a sequence that is complementary to the sequence of the template polynucleotide. The complement polynucleotide can be subjected to nucleic acid analysis, including sequencing and composition analysis.

The phrase “locked nucleic acid” (LNA) refers to a modified RNA nucleotide in which the ribose is modified with an extra bridge connecting the 2′ oxygen and 4′ carbon. A locked nucleic acid can be resistant to cleavage by Exonuclease III.

The phrases “binding pair” and “binding partner” and its variants refers to two molecules, or portions thereof, which have a specific binding affinity for one another and typically can bind to each other in preference to binding to other molecules. The two members of a binding pair are referred to herein as the “first member” and the “second member” respectively. Examples of molecules that function as binding pairs include: biotin (and its derivatives) and their binding partners avidin, streptavidin (and their derivatives); His-tags which bind with nickel, cobalt or copper; cysteine, histidine, or histidine patch which bind Ni-NTA; maltose which binds with maltose binding protein (MBP); lectin-carbohydrate binding partners; calcium-calcium binding protein (CBP); acetylcholine and receptor-acetylcholine; protein A and binding partner anti-FLAG antibody; GST and binding partner glutathione; uracil DNA glycosylase (UDG) and ugi (uracil-DNA glycosylase inhibitor) protein; antigen or epitope tags which bind to antibody or antibody fragments, particularly antigens such as digoxigenin, fluorescein, dinitrophenol or bromodeoxyuridine and their respective antibodies; mouse immunoglobulin and goat anti-mouse immunoglobulin; IgG bound and protein A; receptor-receptor agonist or receptor antagonist; enzyme-enzyme cofactors; enzyme-enzyme inhibitors; and thyroxine-cortisol. Another binding partner for biotin is a biotin-binding protein from chicken (Hytonen, et al., BMC Structural Biology 7:8).

### Compositions

Provided herein are compositions for immobilizing a complementary sequence of the template polynucleotide in an orientation that is flipped compared to the orientation of the template polynucleotide.

Provided herein are adaptive oligonucleotides comprising at least one functional sequence or site in any combination and in any order, including: a cleavage susceptible site, a cleavage resistant site, a priming sequence, a cross-linking sequence, a triple-strand forming sequence, a restriction endonuclease recognition sequence, and/or a nicking endonuclease recognition sequence. The functional sequence or site permits nucleic acid manipulations, such as cleavage, primer extension, or cross-linking. In some embodiments, the adaptive oligonucleotides that can fold into a secondary structure, such as a U-shaped or hairpin structure. In some embodiments, the adaptive oligonucleotide can function as a primer for primer extension reactions.

In some embodiments, the adaptive oligonucleotides can be joined to the template polynucleotide. The various functional sequence or site on the adaptive oligonucleotide permits various nucleic acid manipulations that can be used to generate a complementary sequence of the template polynucleotide with an orientation that is flipped compared to the template oligonucleotide.

### Orientation

In some embodiments, the 5′ end of the target polynucleotide can be proximal to the solid surface and the 3′ end of the template can be distal to the solid surface (i.e., forward orientation). In some embodiments, the 3′ end of the target polynucleotide can be proximal to the solid surface and the 5′ end of the template can be distal to the solid surface (i.e. flipped orientation). In some embodiments, the forward-oriented and flip-oriented target polynucleotides can be sequenced using the same sequencing reagents. In some embodiments, the forward-oriented or flip-oriented target polynucleotide can be joined to an adaptive oligonucleotide.

### Intervening Adaptive Oligonucleotides

In some embodiments, a template polynucleotide can be attached directly to a solid surface. In some embodiments, one or more intervening adaptive oligonucleotides can attach a template polynucleotide to the solid surface. For example, an adaptive oligonucleotide can be attached to the solid surface and a template can be attached to the adaptive oligonucleotide. In some embodiments, an adaptive oligonucleotide can be joined to a target polynucleotide by a ligase. In some embodiments, an adaptive oligonucleotide can be attached to a solid surface, and the 5′ end of the target polynucleotide can be attached to the adaptive oligonucleotide (the 5′ end of the template is proximal to the solid surface). In some embodiments, an adaptive oligonucleotide can be attached to a solid surface, and the 3′ end of the target polynucleotide can be attached to the adaptive oligonucleotide (the 3′ end of the template is proximal to the solid surface). In some embodiments, an adaptive oligonucleotide can be a nucleic acid or nucleic acid analog. In some embodiments, an adaptive oligonucleotide can be a single-stranded nucleic acid. In some embodiments, an adaptive oligonucleotide has a terminal 5′ phosphate group. In some embodiments, an adaptive oligonucleotide has a terminal 3′ OH group. In some embodiments, an adaptive oligonucleotide has a blocking group on the terminal 5′ end or the terminal 3′ end. In some embodiments, a blocking group can inhibit joining an adaptive oligonucleotide to another nucleic acid or to a chemical compound. In some embodiments, a blocking group can inhibit nucleotide polymerization with a polymerase. In some embodiments, an adaptive oligonucleotide can be attached to a solid surface with a linker. In some embodiments, an adaptive oligonucleotide can function as a nucleic acid primer for primer extension reactions.

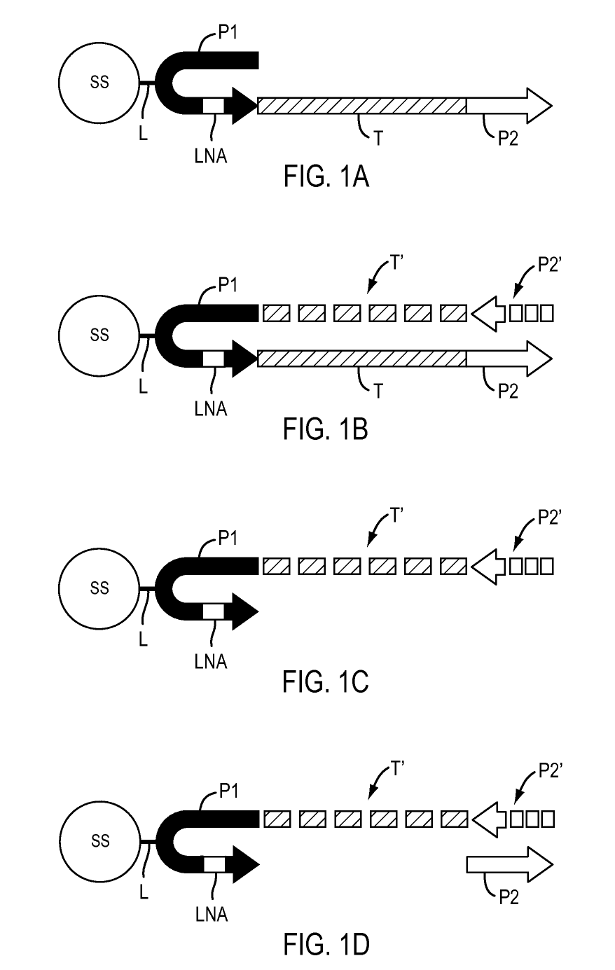

In some embodiments, an adaptive oligonucleotide can fold into a secondary structure, such as a U-shaped or hairpin structure (FIGS. 1A-D).

In some embodiments, an adaptive oligonucleotide includes at least one functional sequence or site in any combination and in any order, including: a cleavage susceptible site, a cleavage resistant site, a priming sequence, a cross-linking sequence, a triple-strand forming sequence, a restriction endonuclease recognition sequence, and/or a nicking endonuclease recognition sequence.

In some embodiments, an adaptive oligonucleotide includes one or more sequences, linkages, or bases, that are resistant or susceptible to cleavage by heat, light, chemical compound, or an enzyme. In some embodiments, an adaptive oligonucleotide can include a nucleic acid linkage that is resistant to cleavage by an exonuclease. For example, the exonuclease-resistant nucleic acid linkage can be a locked nucleic acid (LNA), which is resistant to cleavage by Endonuclease III. In some embodiments, an adaptive oligonucleotide can include a base or linkage that is susceptible to cleavage by an endonuclease. For example, the endonuclease-susceptible base can be one or more inosine bases, which is susceptible to cleavage by Endonuclease V. In another example, the endonuclease-susceptible site is an apurinic tetrahydrofuran site (THF), which is susceptible to cleavage by Endonuclease IV. In some embodiments, an adaptive oligonucleotide can include one or more cleavage sequences (CS). For example, the cleavage sequence can include at least one 2′-deoxyuridine residue. In some embodiments, a 2′-deoxyuridine residue can be cleaved with a uracil DNA glycosylase (UDG).

In some embodiments, the adaptive oligonucleotide includes one or more restriction endonuclease recognition sequences. In some embodiments, the restriction endonuclease recognition sequence includes sequences that are recognized by a restriction enzyme that cleaves within a recognition sequence, or a short distance from the recognition sequence, or cleaves at a remote distance from the recognition sequence. In some embodiments, the restriction endonuclease recognition sequences include type I, type II (e.g., type II, type IIs, type IIG), type III, and type IV sequences. In some embodiments, the restriction endonuclease recognition sequence can be a FokI, AlwI, EcoP151, Eco571, MmeI, or BcgI sequence.

In some embodiments, an adaptive oligonucleotide includes one or more recognition sequences for a nicking endonuclease enzyme. For example, a nicking enzyme recognition site includes Nt.BstNBI, Nb.BsrDI, Nb.BtsI, Nt.AlwI, Nb.BbvCI, Nt.BbvCI, BbvCI, and Nb.BsmI.

In some embodiments, an adaptive oligonucleotide includes one or more sequences that mediate nucleic acid triple strand formation. For example, a triplex-forming oligonucleotides (XO) can include an G/A motif that forms triplet strands with a template polynucleotide having a homopurine tract (e.g., 5′-AAA-3′) or an A/T tract. In some embodiments, an adaptive oligonucleotide includes a cross-linking sequence (XS) comprising a 5′-AAA-poly(pyrimidine)-AATT-3′ sequence. In some embodiments, a cross-linking sequence (XS) can mediate triple strand formation. In some embodiments, a second adaptive oligonucleotide having a sequence that is complementary to the adaptive oligonucleotide (XS′) comprises a 3′-TTT-poly(purine)-TTAA-5′ sequence. In some embodiments, a triplex-forming oligonucleotides (XO) comprises a G/A motif. In some embodiments, a triplex-forming oligonucleotides (XO) can be conjugated with any reactive group that can undergo alkylation with nucleic acids to form triple strands. In some embodiments, an alkylation reaction can be conducted under suitable conditions, such as elevated temperatures (e.g., about 85-97° C.). In some embodiments, a triplex-forming oligonucleotide (XO) can be conjugated with any reactive group that can cross-link nucleic acids, such as psoralen (Wang 1995 Journal of Biol. Chem. 270:22595-22601), bromoacetyl (Povsic 1992 Journal of Am. Chem. Soc. 114:5934-5941), nitrogen mustard residues (Kutyavin 1993 Journal of Am. Chem. Soc. 115:9303-9304), or transplatin adducts (Colombier 1996 Nucleic Acids Research 24:4519-4524). Examples of triplex-forming oligonucleotides, and the sequences of the template duplexes can be found in Lukhtanov 1997 Nucleic Acids Research 25:5077-5084. In some embodiments, a triplex-forming oligonucleotide (XO) can include backbone modifications having peptide nucleic acids or N3′>P5′ phosphoramidates which can form triple strands. In some embodiments, a triplex-forming oligonucleotide (XO) can include alkylating groups, such as for example cyclopropapyrroloindole (CPI) (Lukhtanov 1996 Nucleic Acids Research 24:683-687) or N5-methyl-CPI (MCPI) (Lukhtanov 1997 Journal of Am. chem. Soc. 119:6214-6225). In some embodiments, a triplex-forming oligonucleotide (XO) can be subjected to an alkylation reaction to chemically cross-link three DNA strands.

In some embodiments, an adaptive oligonucleotide can be joined to an aminated oligonucleotide. An aminated oligonucleotide can bind to a solid surface.

### Solid Surfaces

In some embodiments, a template polynucleotide or complement polynucleotide can be attached to a solid surface. In some embodiments, a solid surface can be a planar surface, as well as concave, convex, or any combination thereof. In some embodiments, a solid surface can be a bead, particle, microparticle, sphere, filter, or gel. In some embodiments, a surface includes the inner walls of a capillary, a channel, a well, groove, channel, reservoir. In some embodiments, a surface can include texture (e.g., etched, cavitated or bumps). In some embodiments, a surface can be non-porous. In some embodiments, a surface can be made from materials such as glass, borosilicate glass, silica, quartz, fused quartz, mica, polyacrylamide, plastic polystyrene, polycarbonate, polymethacrylate (PMA), polymethyl methacrylate (PMMA), polydimethylsiloxane (PDMS), silicon, germanium, graphite, ceramics, silicon, semiconductor, high refractive index dielectrics, crystals, gels, polymers, or films (e.g., films of gold, silver, aluminum, or diamond). In some embodiments, immobilized polynucleotides and/or adaptive oligonucleotides can be arranged in a random or ordered array on a surface. An ordered array includes rectilinear and hexagonal patterns.

### Linkers

A suitable linker can be used to attach a solid surface to a template polynucleotide, complement polynucleotide, or adaptive oligonucleotide. Selection of a suitable linker may depend upon the type of linking chemistry available on a solid surface and the type of chemical groups available on a template polynucleotide, complement polynucleotide, or adaptive oligonucleotide. Selecting a suitable linker and implementing linkage of a solid surface to any type of polynucleotide is well known in the art. Any linking chemistry can be used to attach a solid surface to a template polynucleotide, complement polynucleotide, or adaptive oligonucleotide. In some embodiments, a suitable linker can attach a template polynucleotide, complement polynucleotide, or adaptive oligonucleotide, to a solid surface via covalent, non-covalent, ionic bonding, hydrophobic interaction, or any combination thereof. Examples of non-covalent attachment includes: ionic, hydrogen bonding, dipole-dipole interactions, van der Waals interactions, ionic interactions, and hydrophobic interactions. In some embodiments, examples of non-covalent attachment includes: nucleic acid hybridization, protein aptamer-target binding, electrostatic interaction, hydrophobic interaction, non-specific adsorption, and solvent evaporation. A suitable linker can be a cleavable, self-cleavable, or fragmentable linker. A suitable linker can be cleavable or fragmentable using temperature, enzymatic activity, chemical agent, and/or electromagnetic radiation. A suitable linker attachment can be reversible. A suitable linker can be rigid or flexible. A suitable linker can be linear, non-linear, branched, bifunctional, trifunctional, homofunctional, or heterofunctional. Many cleavable, and bifunctional (both homo- and hetero-bifunctional) linkers with varying lengths are available commercially. A suitable linker can have pendant side chains and/or pendant functional groups. A suitable linker can be resistant to heat, salts, acids, bases, light, chemicals, or shearing forces or flow. In some embodiments, a suitable linker does not interfere with any reactions used for strand flipping or sequencing. In some embodiments, a suitable linker can be a binding pair, such as biotin/streptavidin.

### Adaptive Oligonucleotides Having Secondary Structures

Provided here are immobilized template polynucleotides comprising a solid surface attached to an adaptive oligonucleotide which is joined to a template polynucleotide (T). In some embodiments, an adaptive oligonucleotide comprises a first priming sequence (P1). In some embodiments, an adaptive oligonucleotide can fold into a secondary structure, such as a U-shaped or hairpin structure (FIG. 1A). In some embodiments, the 3′ terminal end of an adaptive oligonucleotide can be joined to a template polynucleotide. In some embodiments, the adaptive oligonucleotide comprises a nucleic acid linkage (e.g., locked nucleic acid) that is resistant to cleavage by an exonuclease (e.g., Exonuclease III). In some embodiments, an adaptive oligonucleotide can include a base (e.g., inosine) that is susceptible to cleavage by an endonuclease (e.g., Endonuclease V). In some embodiments, a template polynucleotide includes a second priming sequence (e.g., P2).

Provided herein is an immobilized complement polynucleotide, comprising a solid surface attached to an adaptive oligonucleotide which is joined to a complement polynucleotide (T′). In some embodiments, an adaptive oligonucleotide comprises a first priming sequence (P1). In some embodiments, an adaptive oligonucleotide can fold into a secondary structure, such as a U-shaped or hairpin structure (FIG. 1D). In some embodiments, the 5′ terminal end of an adaptive oligonucleotide can be joined to a complement polynucleotide. In some embodiments, an adaptive oligonucleotide comprises a nucleic acid linkage (e.g., locked nucleic acid) that is resistant to cleavage by an exonuclease (e.g., Exonuclease III). In some embodiments, an adaptive oligonucleotide can include a base (e.g., inosine) that is susceptible to cleavage by an endonuclease (e.g., Endonuclease V). In some embodiments, a complement polynucleotide includes a priming sequence (e.g., P2′).

### Triple Strand Formation

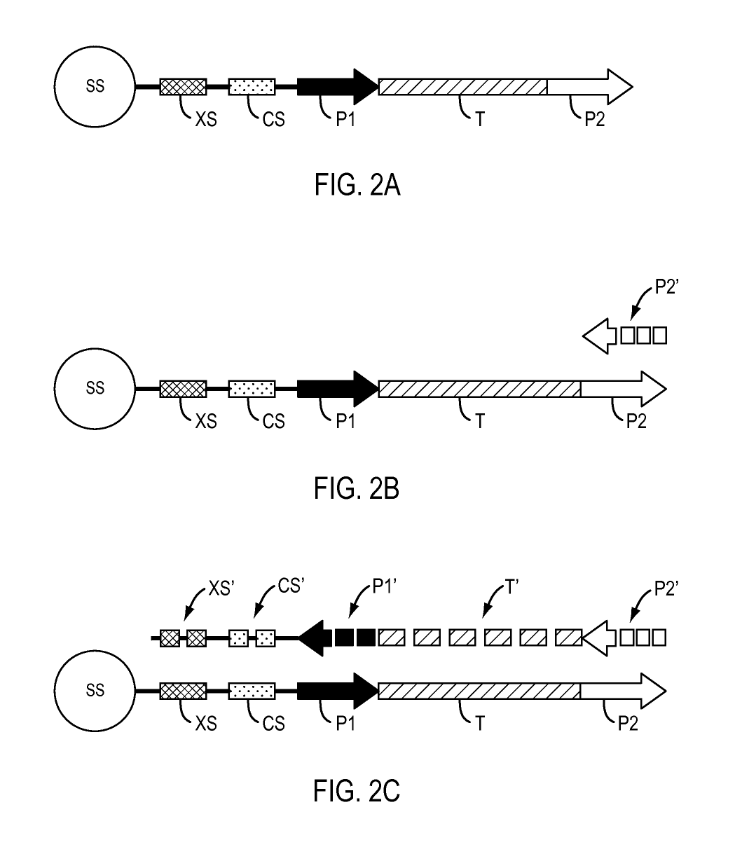

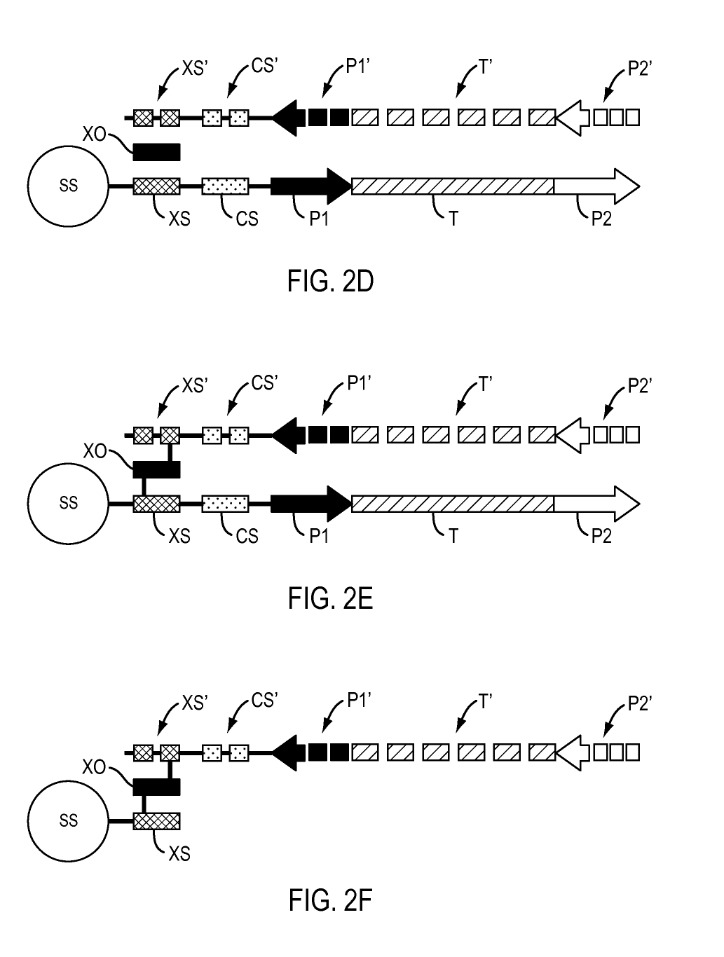

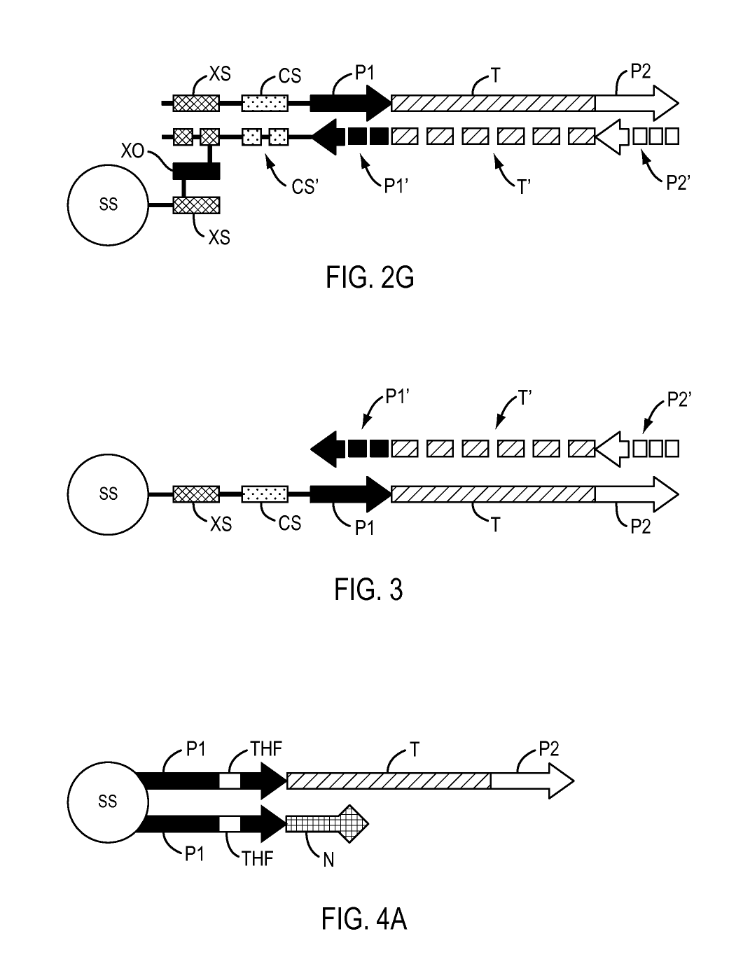

Provided herein is an immobilized template polynucleotide, comprising a solid surface attached to an adaptive oligonucleotide which is joined to a template polynucleotide (T). In some embodiments, an adaptive oligonucleotide comprises a cross-linking sequence (XS) which will form a triple-strand with a second adaptive oligonucleotide having a complementary sequence (XS') and a triplex-forming oligonucleotide (XO) (FIG. 2A). In some embodiments, an adaptive oligonucleotide includes a cleavage sequence (CS). For example, a cleavage sequence (CS) can include at least one 2′-deoxyuridine residue. In some embodiments, a cleavage sequence (CS) can be cleaved with a uracil DNA glycosylase (UDG). In some embodiments, an adaptive oligonucleotide includes a first priming sequence (P1). In some embodiments, the 3′ terminal end of an adaptive oligonucleotide can be joined to the template polynucleotide. In some embodiments, a template polynucleotide includes a second priming sequence (e.g., P2).

Provided herein is an immobilized complement polynucleotide, comprising a solid surface attached to a second adaptive oligonucleotide which is joined to a complement polynucleotide (T′). In some embodiments, a second adaptive oligonucleotide comprises a complementary sequence to the cross-linking sequence (XS') which forms a triple strand with a triplex-forming oligonucleotide (XO) and a cleavage sequence (XS) (FIG. 2F). In some embodiments, a second adaptive oligonucleotide comprises a complementary sequence to the cleavage sequence (CS'). In some embodiments, an adaptive oligonucleotide comprises a complement to a first priming sequence (P1′). In some embodiments, the 5′ terminal end of a second adaptive oligonucleotide can be joined to a template polynucleotide. In some embodiments, a template polynucleotide includes a complement of a second priming sequence (P2′).

### Folded Templates

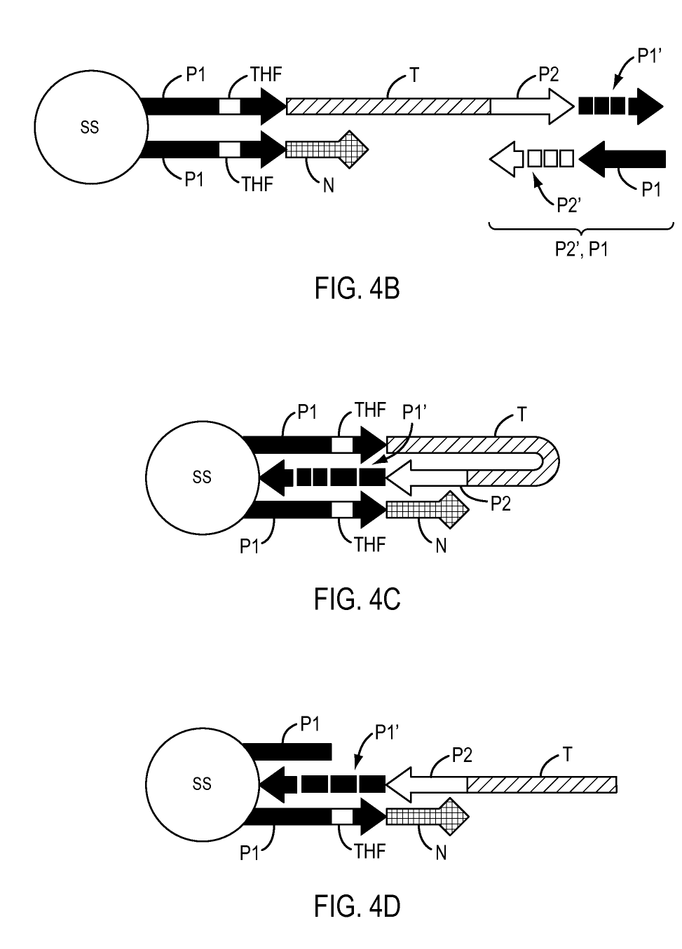

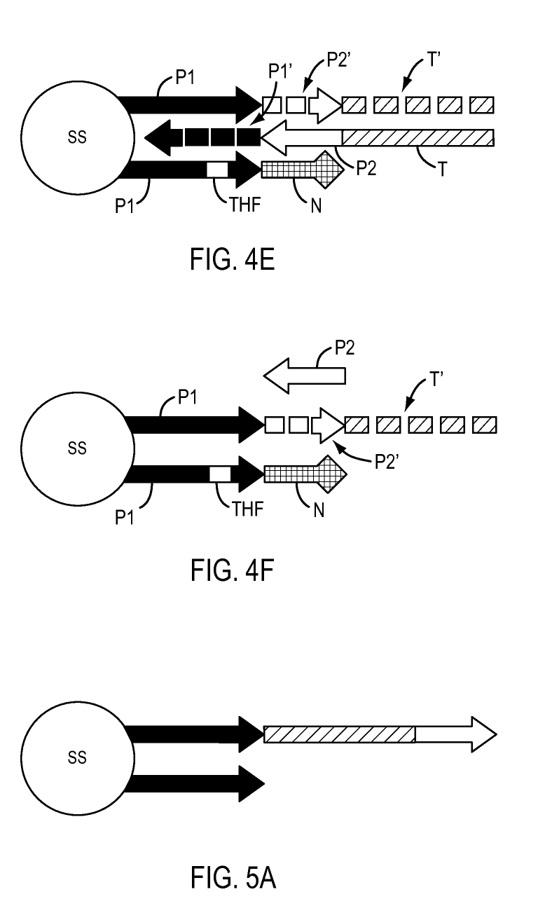

Provided here is a solid surface attached with two different types of adaptive oligonucleotides (FIG. 4A). In some embodiments, a first type of adaptive oligonucleotide comprises a first priming sequence (P1). In some embodiments, a first type of adaptive oligonucleotide comprises an apurinic tetrahydrofuran site (THF) that is susceptible to cleavage by an endonuclease (e.g., Endonuclease IV) (FIG. 4A). In some embodiments, the 3′ terminal end of a first type of adaptive oligonucleotide can be joined to a template polynucleotide. In some embodiments, a template polynucleotide includes a second priming sequence (e.g., P2).

In some embodiments, a second type of adaptive oligonucleotide comprises a first priming sequence (P1). In some embodiments, a second type of adaptive oligonucleotide comprises an apurinic tetrahydrofuran site (THF) that is susceptible to cleavage by an endonuclease (e.g., Endonuclease IV) (FIG. 4A). In some embodiments, the 3′ terminal end of a second type of adaptive oligonucleotide can be joined to an aminated oligonucleotide (N).

Provided here is a solid surface attached with three different nucleic acids. In some embodiments, the first nucleic acid can be a first type of adaptive oligonucleotide comprising a first priming sequence (P1) (FIG. 4D). In some embodiments, the second type of nucleic acid can be an adaptive oligonucleotide comprises a first priming sequence (P1). In some embodiments, a second type of adaptive oligonucleotide comprises an apurinic tetrahydrofuran site (THF) that is susceptible to cleavage by an endonuclease (e.g., Endonuclease IV) (FIG. 4A). In some embodiments, the 3′ terminal end of a second type of adaptive oligonucleotide can be joined to an aminated oligonucleotide (N). In some embodiments, the third nucleic acid can be a second type of adaptive oligonucleotide comprising a complementary sequence to the first priming sequence (P1′). In some embodiments, a third type of adaptive oligonucleotide is joined to a second priming sequence (P2). In some embodiments, a second priming sequence (P2) is joined to a template oligonucleotide (T). In some embodiments, a P1′ sequence in the third type of adaptive oligonucleotide is hybridized to a first priming sequence (P1) which is in a first or the second adaptive oligonucleotide, so that the 3′ end of the third type of adaptive oligonucleotide is proximal to the solid surface.

In some embodiments, a solid surface SS can be attached to one or more adaptive oligonucleotides, where at least one of the adaptive oligonucleotides includes a first priming sequence P1 and a site that is cleavable. In some embodiments, the cleavable site can include one or more uracil residues. In some embodiments, the enzyme that cleaves the cleavable site is a uracil DNA glycosylase (UDG). In some embodiments, at least one of the adaptive oligonucleotides can be attached to a template having a complementary first priming sequence P1′ that permits the template to fold so that the first priming sequence P1 and the complementary first priming sequence P1′ hybridize to each other. In some embodiments, the folded template can be used with a template walking procedure (U.S. Ser. No. 61/424,599, filed Dec. 17, 2010).

### Methods

Provided herein are methods for immobilizing a complementary sequence of the template polynucleotide in an orientation that is flipped compared to the orientation of the template polynucleotide. The flipped orientation of the complementary sequence of the template polynucleotide permits use of the same reagents and conducting the same primer extension or sequencing reactions on the template polynucleotide and the complementary sequence of the template polynucleotide.

Provided herein are methods for immobilizing a polynucleotide, comprising: attaching an adaptive oligonucleotide to a solid surface. In some embodiments, an adaptive oligonucleotide can be joined to a template polynucleotide (T). In some embodiments, an adaptive oligonucleotide includes a first priming sequence (P1). In some embodiments, a template oligonucleotide includes a second priming sequence (P2). In some embodiments, the methods can further comprise: hybridizing a primer (P2′) to a second priming sequence (P2). In some embodiments, the methods can further comprise: extending a P2′ primer with a primer extension reaction to generate a complement polynucleotide. In some embodiments, the primer extension reaction can be conducted with a template-dependent DNA polymerase and nucleotides. In some embodiments, the DNA polymerase can be a mesophilic or thermophilic enzyme. In some embodiments, the nucleotides can be labeled with a reporter moiety (e.g., a fluorophore) or can be unlabeled. In some embodiments, the primer extension reaction can be conducted with an oligonucleotide ligation reaction, such as the SOLiD™ primer extension reactions that are used for sequencing a template. In some embodiments, the oligonucleotide ligation reaction can be conducted with unlabeled oligonucleotide probes or with labeled oligonucleotide probes (e.g., fluorophore-labeled probes for SOLiD™ sequencing, see WO 2006/084132). In some embodiments, a complement polynucleotide can be joined to an immobilized adaptive oligonucleotide, so as to generate an immobilized complement polynucleotide. In some embodiments, the orientation of an immobilized complement polynucleotide can be flipped compared to the orientation of the immobilized template polynucleotide.

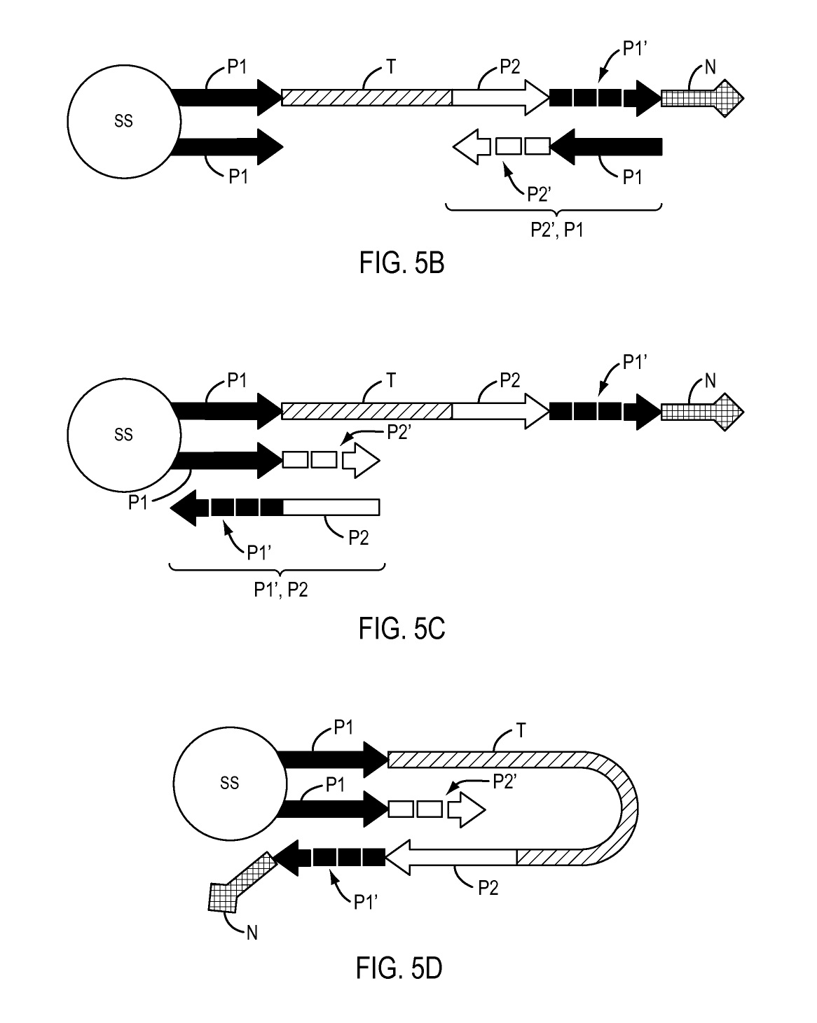

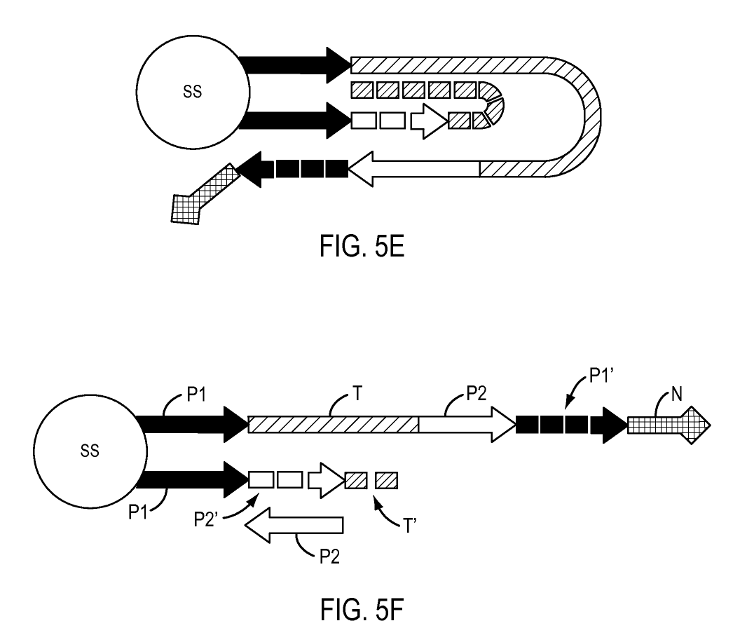

The methods can further comprise: hybridizing a hybrid/chimeric primer that includes two or more different primer sequences (FIGS. 4B and 5B). For example, a hybrid primer can include a P1 and P2′ sequence, or a P1′ and P2 sequence. The methods can further comprise: extending a hybrid primer with a primer extension reaction to generate a complement polynucleotide. The orientation of the complement polynucleotide, so generated by primer extension from the hybrid primer, can be flipped compared to the orientation of the immobilized template polynucleotide.

In some embodiments, an adaptive oligonucleotide includes a sequence, site, or linkage that permits removal of the template oligonucleotide and leaves the complement polynucleotide intact. For example, an adaptive oligonucleotide includes a cleavage susceptible site, a cleavage resistant site, a priming sequence, a cross-linking sequence, a triple-strand forming sequence, a restriction endonuclease recognition sequence, and/or a nicking endonuclease recognition sequence. Thus, removal of a template polynucleotide includes reacting an adaptive oligonucleotide with an enzyme or chemical compound that cleaves the cleavage susceptible site or sequence, or reacting the adaptive oligonucleotide with an enzyme or chemical compound that does not cleave the resistant cleavage site or sequence.

In some embodiments, after the template polynucleotide is removed, the complement polynucleotide remains immobilized to the solid surface and can serve as a template for sequencing reactions. In some embodiments, the orientation of the immobilized complement polynucleotide can be flipped compared to the orientation of the immobilized template polynucleotide.

Provided herein are methods for sequencing a template polynucleotide, or for sequencing a complement polynucleotide. In some embodiments, a sequencing reaction can be conducted on an immobilized template polynucleotide or on an immobilized complement polynucleotide. In some embodiments, a template polynucleotide and a complement polynucleotide can be sequenced using the same reagents. Any type of sequencing reactions can be used, including sequencing-by-ligation (e.g., WO 2006/084132, SOLiD™ by Applied Biosystems, now part of Life Technologies), sequencing-by-synthesis using a template-dependent DNA polymerase (e.g., SOLEXA by Illumina) and pyrophosphate sequencing by 454 Life Sciences.

### Methods Using Adaptive Oligonucleotides Having Secondary Structure:

Provided herein are methods for immobilizing a template oligonucleotide in a first orientation, and immobilizing a complementary sequence of the template polynucleotide in an orientation that is flipped compared to the orientation of the template polynucleotide. In some embodiments, the method comprises the steps: (a) attaching a solid surface to an oligonucleotide (e.g., adaptive oligonucleotide) which is joined to a template polynucleotide, (i) wherein the single-stranded oligonucleotide includes a first priming sequence P1 and includes a nucleic acid base or linkage that is susceptible or resistant to enzymatic cleavage and forms a secondary structure (e.g., FIG. 1A) that is a hairpin or U-shaped secondary structure, and (ii) wherein the template polynucleotide includes a P2 priming sequence; (b) hybridizing a P2′ primer to the P2 priming sequence; (c) extending the P2′ primer with a primer extension reaction to generate a complement polynucleotide; (d) joining the complement polynucleotide to the single-stranded oligonucleotide thereby immobilizing the complement polynucleotide to the solid surface; and (e) conducting an enzymatic reaction on the susceptible or resistant enzyme cleavage site to remove the template polynucleotide from the solid surface so as to generate an immobilized complement polynucleotide.

In some embodiments, the immobilized complement polynucleotide of step (e) has an orientation that is flipped compared to the orientation of the immobilized template polynucleotide in step (a). In some embodiments, the methods further comprise: determining the sequence of the immobilized complement polynucleotide. In some embodiments, the nucleic acid base that is susceptible to enzymatic cleavage is an inosine base. In some embodiments, the enzymatic cleavage is conducted with endonuclease V. In some embodiments, the linkage that is resistant to enzymatic cleavage is a locked nucleic acid LNA. In some embodiments, the enzymatic cleavage is endonuclease III.

In some embodiments, the methods comprise: generating a U-shape adaptive oligonucleotide joined to a template polynucleotide, wherein the U-shaped adaptive oligonucleotide includes a 5′ phosphate end, a 3′ end, and at least one inosine base. In some embodiments, the U-shaped adaptive oligonucleotide can be attached to a solid support. In some embodiments, the template polynucleotide includes a second primer site P2. In some embodiments, the methods comprise: adding a second primer P2′ that is complementary to the 3′ end of the template polynucleotide, and extending the second primer P2′ with a primer extension reaction to generate a complement polynucleotide T′; ligating the complement polynucleotide to the U-shaped adaptive oligonucleotide; cleaving the template nucleotide sequence T; and determining the sequence of the complement polynucleotide.

One exemplary method using a U-shaped adaptive oligonucleotide is depicted schematically in FIGS. 1A-1D. In FIG. 1A, a U-shaped adaptive oligonucleotide comprising a first primer site P1 can be attached to a solid support SS by a linker L. The U-shaped adaptive oligonucleotide comprises at least one locked nucleic acid LNA. A template polynucleotide T can be joined to the U-shaped adaptive oligonucleotide. The template polynucleotide can include a second primer site P2. As shown in FIG. 1A and the subsequent figures, primer sites and primers are shown with arrows to show the 5′ to 3′ direction.

The template polynucleotide T can be sequenced to generate sequencing data in the 5′ to 3′ direction of the template sequence T. A complement to the second primer site P2′, nucleotides, polymerase, and ligase can be added to generate the template sequence complement T′ (FIG. 1B).

The template polynucleotide T may be digested from the 3′ end with Exonuclease III, which cleaves the 3′ end of the double-stranded nucleic acid to within 1 nucleotide of the locked nucleic acid LNA. After washing (an optional step), the complement polynucleotide T′ and the second primer sequence complement P2′ can be left attached to the U-shaped adaptive oligonucleotide (FIG. 1C).

A second primer site P2 can be hybridized to the second primer sequence complement P2′ followed by sequencing the template sequence complement T′ in the 5′ to 3′ direction (FIG. 1D).

In the method shown in FIGS. 1A-1D, the template polynucleotide T and the complement polynucleotide T′ can be sequenced.

In some embodiments, the at least one locked nucleic acid may be replaced with at least one inosine base. The adaptive oligonucleotide can be reacted with Endonuclease V. The Endonuclease V can cleave a double-stranded nucleic acid at the 3′ end of the inosine base. The template polynucleotide, so reacted with Endonuclease V, can be washed away, leaving the complement polynucleotide immobilized. The immobilized complement polynucleotide can be sequenced.

### Methods Using Triple-Strand Formation

Provided herein are methods for immobilizing a template oligonucleotide in a first orientation, and immobilizing a complementary sequence of the template polynucleotide in an orientation that is flipped compared to the orientation of the template polynucleotide. In some embodiments, the method comprises the steps: (a) attaching a solid surface to an oligonucleotide (e.g., adaptive oligonucleotide) which is joined to a template polynucleotide, (i) wherein the single-stranded oligonucleotide includes a first priming sequence P1 and includes an enzyme-cleavable base CS and includes a nucleotide sequence that mediates triple-strand formation XS (FIG. 2A), and (ii) wherein the template polynucleotide includes a P2 priming sequence; (b) hybridizing a P2′ primer to the P2 priming sequence; (c) conducting a primer extension reaction on the P2′ primer to generate a complement polynucleotide; (d) reacting the nucleotide sequence that mediates triple-strand formation XS and the complement polynucleotide with a triplex-forming oligonucleotide XO under suitable conditions so as to form a triple strand; and (e) cleaving the enzyme-cleavable base CS with an enzyme to remove the template polynucleotide from the solid surface so as to generate an immobilized complement polynucleotide.

In some embodiments, the immobilized complement polynucleotide of step (e) has an orientation that is flipped compared to the orientation of the immobilized template polynucleotide in step (a). In some embodiment, the method further comprises: determining the sequence of the immobilized complement polynucleotide. In some embodiments, the sequence that mediates triple-strand formation XS comprises 5′ AAA-poly(pyrimidine)-AATT 3′. In some embodiments, the triplex-forming oligonucleotide XO comprises a G/A motif. In some embodiments, the enzyme-cleavable base CS is a 2′-deoxyuridine. In some embodiments, the cleaving of step (e) is conducted with a uracil DNA glycosylase (UDG).

In some embodiments, the triple strands can be generated using the sequence that mediates triple-strand formation and/or the triplex-forming oligonucleotides described in Lukhtanov et al., “Minor groove DNA alkylation directed by major groove triplex forming oligodeoxyribonucleotides,” Nucleic Acids Research, Vol. 25, No. 24, pp. 5077-5084 (1997).

One exemplary method using triplex formation is schematically depicted in FIGS. 2A-2G. In FIG. 2A, a template polynucleotide T can be joined to an adaptive oligonucleotide having a first primer sequence P1. The template polynucleotide can include a second primer sequence P2. The adaptive oligonucleotide can be attached to a solid support SS via a cross-linking sequence XS and a cleavage sequence CS. In some embodiments, a cross-linking sequence XS comprises a 5′-AAA-poly(pyrimidine)-AATT-3′ sequence to which a triplex forming oligonucleotide, or cross-linking oligonucleotide XO, can bind after duplex formation with a 3′-TTT-poly(purine)-TTAA-5′ sequence. In some embodiments, the cross-linking sequence complement XS′ includes 3′-TTT-poly(purine)-TTAA-5′. In some embodiments, a cleavage sequence CS includes at least one 2′-deoxyuridine residue.

In some embodiments, a second primer site complement P2′ can be hybridized to the second primer site P2 and the second primer site complement P2′ may be extended using a polymerase and nucleotides to generate a nucleic acid comprising a template sequence complement T′, a first primer site complement P1′, a cleavage sequence complement CS', and a cross-linking sequence complement XS′ (FIGS. 2B and 2C).

In some embodiments, a cross-linking oligonucleotide XO can be hybridized to the cross-linking sequence XS and the cross-linking sequence complement XS′ to form a triple stranded nucleic acid, (FIGS. 2D and 2E). In some embodiments, the cross-linking oligonucleotide XO comprises a triplex forming oligonucleotide. In some embodiments, the cross-linking oligonucleotide comprises the modified sequence (+)MCPI-DPI-X8-5′-AGGAGAGGAGAGAGGAAGAGAAGG-3′-X8-DPI-(+)MCPI, which is disclosed, for example, as triplex forming oligonucleotide 23 in Lukhtanov et al., “Minor groove DNA alkylation directed by major groove triplex forming oligodeoxyribonucleotides,” Nucleic Acids Research, Vol. 25, No. 24, pp. 5077-5084 (1997). In some embodiments, after cross-linking, the cleavage sequence CS can be cleaved using uracil DNA glycosylase (UDG) to generate an immobilized complement polynucleotide T′ in an orientation that is flipped compared to the orientation of the template polynucleotide T (FIG. 2F). In some embodiments, the complement polynucleotide T′ can be sequenced (FIG. 2G).

As shown in FIG. 3, template sequence T may be sequenced by hybridizing a first primer site complement P1′.

### Methods Using Apurinic Tetrahydrofuran Sites

Provided herein are methods for immobilizing a template oligonucleotide in a first orientation, and immobilizing a complementary sequence of the template polynucleotide in an orientation that is flipped compared to the orientation of the template polynucleotide. In some embodiments, the method comprises the steps: (a) attaching a solid surface to an oligonucleotide (e.g., adaptive oligonucleotide) which is joined to a template polynucleotide, (i) wherein the single-stranded oligonucleotide includes a first priming sequence P1 and includes a first enzyme-cleavable site and (ii) wherein the template polynucleotide includes a second priming sequence P2, and wherein the second single-stranded oligonucleotide is joined to an aminated oligonucleotide (FIG. 4A), wherein the second single-stranded oligonucleotide includes a second enzyme-cleavable base; (b) hybridizing a P1/P2′ hybrid primer to the P2 priming sequence; (c) extending the second priming sequence P2 so as to generate an extended template polynucleotide having a first priming sequence P1, first enzyme-cleavable base, a template polynucleotide sequence, a second priming sequence P2, and an extended P1′ sequence; (d) folding the extended template polynucleotide on itself, so as to hybridize the P1 sequence with the P1′ sequence; (e) removing the first enzyme-cleavable base with an enzymatic reaction so as to leave the first single-stranded oligonucleotide having the first primer sequence P1 immobilized to the bead, and so as to leave the extended template polynucleotide hybridized to the first primer sequence P1 that is immobilized to the bead.

In some embodiments, the extended template polynucleotide of step (e) has an orientation that is flipped compared to the orientation of the immobilized template polynucleotide in step (a). In some embodiments, the method further comprises determining the sequence of the immobilized complement polynucleotide. In some embodiments, the first enzyme-cleavable base is an apurinic tetrahydrofuran site. In some embodiments, the enzyme that cleaves the apurinic tetrahydrofuran site is endonuclease IV.

In some embodiments, paired end sequencing can be conducted by extending the second primer site P2. An example of such a method is schematically depicted in FIGS. 4A-4F.

In FIG. 4A, a template polynucleotide T is attached to an adaptive oligonucleotide having a first primer site P1. The adaptive oligonucleotide can be attached to the solid support SS by a linker L. The first primer site P1 can include an apurinic tetrahydrofuran site THF that can be cut by Endonuclease IV. Alternatively, the first primer site P1 can comprise a nicking enzyme recognition site that can be cut by a nicking enzyme.

In some embodiments, the solid support SS can comprise a plurality of attached adaptive oligonucleotides having, for example, first primer sites P1. The adaptive oligonucleotides that do not have a template sequence T bound thereto can optionally be joined to an aminated oligonucleotide N, which may, for example, improve binding to a solid surface.

In some embodiments, the second primer site P2 can be extended by a hybrid primer P2′P1 comprising a second primer site complement P2′ and first primer site P1 to create a first primer site complement P1′ extension (FIG. 4B).

In some embodiments, the temperature may be increased to melt the double-stranded nucleic acid. Upon cooling, the first primer site complement P1′ can hybridize to the first primer site P1, to form a secondary structure (e.g., a hairpin) (FIG. 4C).

In some embodiments, after a secondary structure formation, Endonuclease IV can be used to cleave the tetrahydrofuran site THF (FIG. 4D). The P1 primer can be used to sequence the template polynucleotide T (FIG. 4E).

In some embodiments, a second primer P2 (FIG. 4F) can be used to sequence the P1 primer site.

In some embodiments, both the second primer site P2 and the at least one first primer site P1 that does not have the template sequence T attached thereto (FIG. 4D) can be extended, to generate a template polynucleotide T forming a hairpin with at least one first primer site P1 that is not attached to the template sequence T.

In at least one embodiment, the at least one first primer site P1 may be replaced with a P4 sequence. The P4 sequence may, for example, comprise the sequence 5′-/5AmMC6 GCG GTC ACG CTG CGC GUA ACC AGC ̂Cac tgc CAC A THF CCA CTA CGC CTC CGC TTT CCT CTC TA-3′.

### Paired End Sequencing

Provided herein are methods for paired end sequencing using unused primer sites on the solid support. Provided herein are methods for immobilizing a template oligonucleotide in a first orientation, and immobilizing a complementary sequence of the template polynucleotide in an orientation that is flipped compared to the orientation of the template polynucleotide. In some embodiments, a template nucleic acid sequence T is attached to an adaptive oligonucleotide having a first primer site P1. The adaptive oligonucleotide can be attached to a solid support SS. The solid support SS can comprise a plurality of first primer sites P1, some of which are not joined to a template polynucleotide T (FIG. 5A).

In some embodiments, a hybrid primer P2′P1 can be used to generate an extended polynucleotide having a P1 and P2′ sequence (FIG. 5B). An aminated oligonucleotide N can be joined to the first primer site complement P1′.

In some embodiments, the unused first primer sites P1 may be extended to include a second primer site complement P2′ using a primer P1′P2 (FIG. 5C).

In some embodiments, the nucleic acids can be denatured by heating. In some embodiments, the nucleic acids can be cooled to permit hairpin formation. For example, the hairpin can include the first primer site P1, the template polynucleotide T, the second primer site P2, and the complement first primer site P1′. In the hairpin, the P1′-P2 sequence can hybridize with the immobilized P1-P2′ sequence (FIG. 5D).

In some embodiments, a polymerase and nucleotides can be used in a primer extension reaction to generate a complement polynucleotide T′ (FIG. 5E). The double-stranded nucleic acid can be denatured with heat. The complement polynucleotide T′ can be sequenced using a second primer site P2, polymerase, and nucleotides (FIG. 5F).

### Kits

Provided herein are kits for immobilizing a template oligonucleotide in a first orientation, and immobilizing a complementary sequence of the template polynucleotide in an orientation that is flipped compared to the orientation of the template polynucleotide. In some embodiments, the kits comprise any combination of: adaptive oligonucleotides; adaptive oligonucleotides that form a secondary structure (e.g., hairpin or U-shaped structure); adaptive oligonucleotides having any combination of a cleavage susceptible site (e.g., inosine, apurinic tetrahydrofuran site, 2′-deoxyuridine residue), a cleavage resistant site (e.g., locked nucleic acids), a priming sequence, a cross-linking sequence, a triple-strand forming sequence, a restriction endonuclease recognition sequence, and/or a nicking endonuclease recognition sequence; nucleic acid cleavage enzymes (e.g., endonucleases III or V, exonucleases); polymerases (e.g., DNA polymerase); nucleotides; ligase; primers having P1, P1′, P2, or P2′ sequences; hybrid primers having any combination of P1, P1′, P2, and/or P2′ sequences; triplex-forming oligonucleotides; reagents for cross-linking nucleic acids (e.g., for generating triple strands); aminated oligonucleotides; beads, flow cell or similar solid surfaces; and/or reagents for linking the beads or flow cell to the adaptive oligonucleotides.

While the principles of the present teachings have been described in connection with specific embodiments of flip strand sequencing methods and primers, it should be understood clearly that these descriptions are made only by way of example and are not intended to limit the scope of the present teachings or claims. What has been disclosed herein has been provided for the purposes of illustration and description. It is not intended to be exhaustive or to limit what is disclosed to the precise forms described. Many modifications and variations will be apparent to the practitioner skilled in the art. What is disclosed was chosen and described in order to best explain the principles and practical application of the disclosed embodiments of the art described, thereby enabling others skilled in the art to understand the various embodiments and various modifications that are suited to the particular use contemplated. It is intended that the scope of what is disclosed be defined by the following claims and their equivalents.

What is claimed is:

1. An immobilized single-stranded oligonucleotide joined to a template polynucleotide, wherein the single-stranded oligonucleotide includes a first primer sequence (P1), and includes nucleic acid linkage that is resistant to cleavage by an exonuclease, and the single-stranded oligonucleotide forms a secondary structure that is a hairpin or U-shaped secondary structure.

2. An immobilized single-stranded oligonucleotide joined to a template polynucleotide, wherein the single-stranded oligonucleotide includes a first primer sequence (P1), and includes a nucleic acid linkage that is susceptible to cleavage by an endonuclease, and the single-stranded oligonucleotide forms a secondary structure that is a hairpin or U-shaped secondary structure.

3. An immobilized single-stranded oligonucleotide joined to a template polynucleotide, wherein the single-stranded oligonucleotide includes a first primer sequence (P1), and includes an enzyme-cleavable base (CS), and includes a nucleotide sequence that mediates triple-strand formation (XS).

4. An immobilized first single-stranded oligonucleotide joined to a template polynucleotide, wherein the first single-stranded oligonucleotide includes a first primer sequence (P1), and includes a first enzyme-cleavable base, and
an immobilized second single-stranded oligonucleotide joined to an aminated oligonucleotide, wherein the second single-stranded oligonucleotide includes a second enzyme-cleavable base, and
wherein the first and the second single-stranded oligonucleotides are immobilized to the same solid surface.

5. An immobilized first single-stranded oligonucleotide joined to a template polynucleotide, wherein the first single-stranded oligonucleotide includes a first primer sequence (P1), and
an immobilized second single-stranded oligonucleotide comprising a first primer sequence (P1), and
wherein the first and the second single-stranded oligonucleotides are immobilized to the same solid surface.

6. A method for immobilizing a polynucleotide, comprising:
a) attaching a solid surface to a single-stranded oligonucleotide which is joined to a template polynucleotide, (i) wherein the single-stranded oligonucleotide includes a first priming sequence (P1) and includes a nucleic acid base or linkage that is susceptible or resistant to enzymatic cleavage and forms a secondary structure that is a hairpin or U-shaped secondary structure, and (ii) wherein the template polynucleotide includes a P2 priming sequence;
b) hybridizing a P2′ primer to the P2 priming sequence;
c) extending the P2′ primer with a primer extension reaction to generate a complement polynucleotide;
d) joining the complement polynucleotide to the single-stranded oligonucleotide thereby immobilizing the complement polynucleotide to the solid surface; and
e) conducting an enzymatic reaction on the susceptible or resistant enzyme cleavage site to remove the template polynucleotide from the solid surface so as to generate an immobilized complement polynucleotide.

7. The method of claim 6, wherein the immobilized complement polynucleotide of step (e) has an orientation that is flipped compared to the orientation of the immobilized template polynucleotide in step (a).

8. The method of claim 6, further comprising determining the sequence of the immobilized complement polynucleotide.

9. The method of claim 6, wherein the nucleic acid base that is susceptible to enzymatic cleavage is an inosine base and the enzymatic cleavage is conducted with endonuclease V.

10. The method of claim 6, wherein the linkage that is resistant to enzymatic cleavage is a locked nucleic acid (LNA) and the enzymatic cleavage is endonuclease III.

11. A method for immobilizing a polynucleotide, comprising:
a) attaching a solid surface to a single-stranded oligonucleotide which is joined to a template polynucleotide, (i) wherein the single-stranded oligonucleotide includes a first priming sequence (P1) and includes an enzyme-cleavable base (CS) and includes a nucleotide sequence that mediates triple-strand formation (XS), and (ii) wherein the template polynucleotide includes a P2 priming sequence;
b) hybridizing a P2′ primer to the P2 priming sequence;
c) conducting a primer extension reaction on the P2′ primer to generate a complement polynucleotide;
d) reacting the nucleotide sequence that mediates triple-strand formation (XS) and the complement polynucleotide with a triplex-forming oligonucleotide (XO) under suitable conditions so as to form a triple strand;
e) cleaving the enzyme-cleavable base (CS) with an enzyme to remove the template polynucleotide from the solid surface so as to generate an immobilized complement polynucleotide.

12. The method of claim 11, wherein the immobilized complement polynucleotide of step (e) has an orientation that is flipped compared to the orientation of the immobilized template polynucleotide in step (a).

13. The method of claim 11, further comprising determining the sequence of the immobilized complement polynucleotide.

14. The method of claim 11, wherein the sequence that mediates triple-strand formation (XS) comprises 5′ AAA-poly(pyrimidine)-AATT 3′.

15. The method of claim 11, wherein the triplex-forming oligonucleotide (XO) comprises a G/A motif.

16. The method of claim 11, wherein the enzyme-cleavable base (CS) is a 2′-deoxyuridine.

17. The method of claim 11, wherein the cleaving of step (e) is conducted with a uracil DNA glycosylase (UDG).

18. A method for immobilizing a polynucleotide, comprising:
a) attaching a solid surface to a first and second single-stranded oligonucleotide, wherein the first single-stranded oligonucleotide is joined to a template polynucleotide, (i) wherein the single-stranded oligonucleotide includes a first priming sequence (P1) and includes a first enzyme-cleavable site and (ii) wherein the template polynucleotide includes a second priming sequence (P2);
and wherein the second single-stranded oligonucleotide is joined to an aminated oligonucleotide, wherein the second single-stranded oligonucleotide includes a second enzyme-cleavable base;
b) hybridizing a P1/P2′ hybrid primer to the P2 priming sequence;
c) extending the second priming sequence (P2) so as to generate an extended template polynucleotide having a first priming sequence (P1), first enzyme-cleavable base, a template polynucleotide sequence, a second priming sequence (P2), and an extended P1′ sequence;
d) folding the extended template polynucleotide on itself, so as to hybridize the P1 sequence with the P1′ sequence;
e) removing the first enzyme-cleavable base with an enzymatic reaction so as to leave the first single-stranded oligonucleotide having the first primer sequence (P1) immobilized to the bead, and so as to leave the extended template polynucleotide hybridized to the first primer sequence (P1) that is immobilized to the bead.

19. The method of claim 18, wherein the extended template polynucleotide of step (e) has an orientation that is flipped compared to the orientation of the immobilized template polynucleotide in step (a).

20. The method of claim 18, further comprising determining the sequence of the immobilized complement polynucleotide.

21. The method of claim 18, wherein the first enzyme-cleavable base is an apurinic tetrahydrofuran site.

22. The method of claim 21, wherein the enzyme that cleaves the apurinic tetrahydrofuran site is endonuclease IV.

23. A kit comprising a single-stranded oligonucleotide having any combination of a cleavage susceptible site, a cleavage resistant site, a priming sequence, a cross-linking sequence, a triple-strand forming sequence, a restriction endonuclease recognition sequence, and/or a nicking endonuclease recognition sequence.

24. The kit of claim 23, wherein the cleavage susceptible site is an inosine base, a 2′ deoxyuridine, or an apurinic tetrahydrofuran site.

25. The kit of claim 23, wherein the cleavage resistant site is a locked nucleic acid.

26. The kit of claim 23, wherein the single-stranded oligonucleotide can form a secondary.

27. The kit of claim 26, wherein the secondary structure that is a hairpin or U-shaped structure.

28. The kit of claim 23, further comprising beads.


SEQUENCE LISTING (3 sequences)

SEQ ID NO: 1 - DNA, 24 bp, Artificial Sequence
  Description of Artificial Sequence Synthetic oligonucleotide
aggagaggagagaggaagagaagg

SEQ ID NO: 2 - DNA, 34 bp, Artificial Sequence
  Description of Artificial Sequence Synthetic oligonucleotide
gcggtcacgctgcgcguaaccagccactgccaca

SEQ ID NO: 3 - DNA, 26 bp, Artificial Sequence
  Description of Artificial Sequence Synthetic oligonucleotide
ccactacgcctccgctttcctctcta